(19) United States
(12) Patent Application Publication
(10) Pub. No.: US 2005/0038252 A1
(43) Pub. Date: Feb. 17, 2005

(54) New difurylglycolic acid esters processes for the preparation thereof as well as the use thereof as pharmaceutical compositions

(75) Inventors: Gerd Morschhaeuser, Biberach (DE); Michael P. Pieper, Biberach (DE); Georg Speck, Ingelheim am Rhein (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/948,427

(22) Filed: Sep. 23, 2004

Related U.S. Application Data
(63) Continuation of application No. 10/379,441, filed on Mar. 4, 2003.
(60) Provisional application No. 60/368,023, filed on Mar. 27, 2002.

(30) Foreign Application Priority Data
Mar. 16, 2002 (DE) 102 11 700.4

(51) Int. Cl. C07D 491/08; C07D 451/02

(52) U.S. Cl. 546/91; 546/127

(57) ABSTRACT

Difurylglycolic acid esters of the formula 1 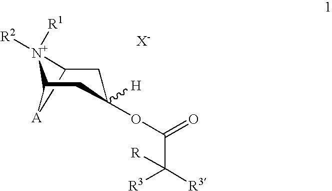
wherein X⁻ and the groups A, R, R¹, R², R³ and R^{3′} may have the meanings given in the claims and in the specification, processes for preparing them and their use for treating asthma, COPD, vagally induced sinus bradycardia, heart rhythm disorders, spasms in the gastrointestinal tract, spasms in the urinary tract or menstrual pain.

### RELATED APPLICATIONS

Benefit of U.S. Provisional Application Ser. No. 60/368,023, filed on Mar. 27, 2002 is hereby claimed.

### FIELD OF THE INVENTION

The present invention relates to new difurylglycolic acid esters of general formula 1 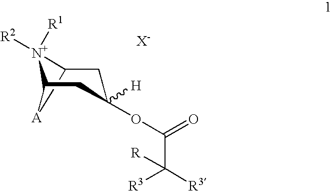
wherein X⁻ and the groups A, R, R¹, R², R³ and R^{3′} may have the meanings given in the claims and in the specification, processes for preparing them and their use as pharmaceutical compositions.

### DESCRIPTION OF THE INVENTION

The present invention relates to compounds of general formula 1 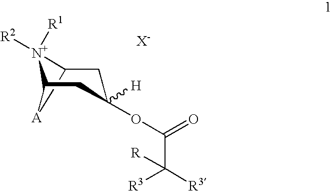
wherein
A denotes a double-bonded group selected from the group consisting of 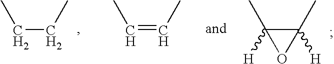
X⁻ denotes an anion with a single negative charge, preferably an anion selected from the group consisting of chloride, bromide, iodide, sulphate, phosphate, methanesulphonate, nitrate, maleate, acetate, citrate, fumarate, tartrate, oxalate, succinate, benzoate and p-toluenesulphonate;
R denotes hydrogen, hydroxy, methyl, ethyl, —CF₃, CHF₂ or fluorine;
R¹ and R² which may be identical or different denote —C₁-C₅-alkyl, which may optionally be substituted by
   —C₃-C₆-cycloalkyl, hydroxy or halogen,
   or
   R¹ and R² together denote a —C₃-C₅-alkylene bridge;
R³ and R³ which may be identical or different denote furyl which may optionally be mono-, di- or trisubstituted by a group selected from the group consisting of —C₁-C₄-alkyl, —C₁-C₄-alkyloxy, hydroxy, —CF₃, —CHF₂, CN, NO₂ or halogen.

Preferred compounds of general formula 1 are those wherein
A denotes a double-bonded group selected from the group consisting of 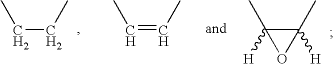
X⁻ denotes an anion with a single negative charge selected from among the chloride, bromide, 4-toluenesulphonate and methanesulphonate, preferably bromide;
R denotes hydroxy, methyl or fluorine;
R¹ and R² which may be identical or different denote methyl, ethyl or fluoroethyl;
R³ and R^{3′} which may be identical or different denote furyl which may optionally be mono- or disubstituted by a group selected from the group consisting of methyl, methyloxy, hydroxy, —CF₃, —CHF₂, fluorine or chlorine.

Particularly preferred are compounds of general formula 1 wherein
A denotes a double-bonded group selected from the group consisting of 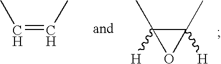
X⁻ denotes an anion with a single negative charge selected from among the chloride, bromide and methanesulphonate, preferably bromide;
R denotes hydroxy, methyl or fluorine, preferably hydroxy;
R¹ and R² which may be identical or different denote methyl or ethyl, preferably methyl;
R³ and R^{3′} which may be identical or different denote furyl which may optionally be mono- or disubstituted by a group selected from the group consisting of —CF₃, —CHF₂ or fluorine.

Of particular importance according to the invention are compounds of general formula 1 wherein
A denotes a double-bonded group selected from the group consisting of 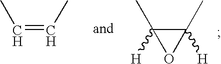
X⁻ denotes bromide;
R denotes hydroxy or methyl, preferably hydroxy;
R¹ and R² which may be identical or different denote methyl or ethyl, preferably methyl;
R³ and R^{3′} which may be identical or different denote furyl which may optionally be mono- or disubstituted by fluorine.

The invention relates to the compounds of formula 1 optionally in the form of the individual optical isomers, mixtures of the individual enantiomers or racemates thereof.

Of particular importance according to the invention are those compounds of general formula 1 wherein the ester substituent at the nitrogen bicyclic group is in the α configuration. These compounds correspond to general formula 1-α

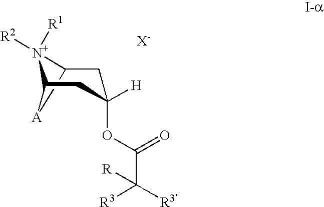

The following compounds are of particular importance according to the invention:
tropenol di-(2-furyl)-glycolate methobromide;
scopine di-(2-furyl)-glycolate methobromide;

The term furyl within the scope of the present invention denotes the group 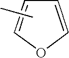
which may be linked in either the 2 or 3 position, based on the position of the oxygen. Both regioisomers are to be regarded as covered by the term furyl. The groups R³ and R^{3′} may either both denote the group 2-furyl or both denote the group 3-furyl. Moreover, the term furyl for the groups R³ and R^{3′} also includes those compounds wherein one of the two groups R³ and R^{3′} denotes 2-furyl and the other denotes 3-furyl. However, particularly preferred compounds according to the invention are those wherein the groups R³ and R^{3′} both denote 2-furyl or both denote 3-furyl, while those compounds in which both groups R³ and R^{3′} denote 2-furyl are of particular significance according to the invention.

The furyl group according to groups R³ and R^{3′} may be unsubstituted or may carry substituents in accordance with the definitions given hereinbefore. The two furyl groups of the groups R³ and R^{3′} may carry identical or different substituents. However, preferred compounds according to the invention are those wherein, if the furyl groups in R³ and R^{3′} carry substituents, both groups carry the same substituents. Particularly preferred are those compounds wherein of the groups R³ and R^{3′} at least one denotes an unsubstituted furyl group. Most preferably, both groups R³ and R^{3′} denote unsubstituted furyl.

The alkyl groups used, unless otherwise stated, are branched and unbranched alkyl groups having 1 to 5 carbon atoms. Examples include: methyl, ethyl, propyl or butyl. The groups methyl, ethyl, propyl or butyl may optionally also be referred to by the abbreviations Me, Et, Prop or Bu. Unless otherwise stated, the definitions propyl and butyl also include all possible isomeric forms of the groups in question. Thus, for example, propyl includes n-propyl and iso-propyl, butyl includes iso-butyl, sec. butyl and tert.-butyl, etc.

The alkylene groups used, unless otherwise stated, are branched and unbranched double-bonded alkyl bridges with 1 to 4 carbon atoms. Examples include: methylene, ethylene, propylene or butylene.

The alkyloxy groups used, unless otherwise stated, are branched and unbranched alkyl groups with 1 to 4 carbon atoms which are linked via an oxygen atom. The following may be mentioned, for example: methyloxy, ethyloxy, propyloxy or butyloxy. The groups methyloxy, ethyloxy, propyloxy or butyloxy may optionally also be referred to by the abbreviations MeO, EtO, PropO or BuO. Unless otherwise stated, the definitions propyloxy and butyloxy also include all possible isomeric forms of the groups in question. Thus, for example, propyloxy includes n-propyloxy and iso-propyloxy, butyloxy includes iso-butyloxy, sec. butyloxy and tert.-butyloxy, etc. The word alkoxy may also possibly be used within the scope of the present invention instead of the word alkyloxy. The groups methyloxy, ethyloxy, propyloxy or butyloxy may optionally also be referred to as methoxy, ethoxy, propoxy or butoxy.

Within the scope of the present invention halogen denotes fluorine, chlorine, bromine or iodine. Unless otherwise stated, fluorine and bromine are the preferred halogens. The group CO denotes a carbonyl group.

As explained hereinafter, the compounds according to the invention may be prepared partly analogously to the methods already known in the art (Diagram 1). The carboxylic acid derivatives of formula 3 are known in the art or may be obtained by methods of synthesis known in the art. If only suitably substituted carboxylic acids are known in the art, the compounds of formula 3 may also be obtained directly from them by acid- or base-catalysed esterification with the corresponding alcohols or by halogenation with the corresponding halogenation reagents. 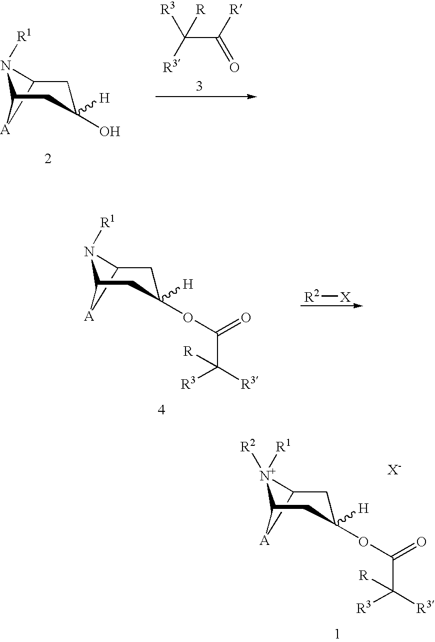
Diagram 1:

As can be seen from Diagram 1, the compounds of formula 2 serve as starting products for preparing the compounds of formula 1. These compounds are known in the prior art.

Starting from the compounds of formula 2 the esters of general formula 4 may be obtained by reaction with the carboxylic acid derivatives of formula 3, wherein R′ denotes for example chlorine or a C₁-C₄-alkyloxy group. When R′ equals C₁-C₄-alkyloxy this reaction may be carried out for example in a sodium melt at elevated temperature, preferably at about 50-150° C., more preferably at about 90-100° C. at low pressure, preferably at below 500 mbar, most preferably at below 75 mbar.

The compounds of formula 4 thus obtained may be converted into the target compounds of formula 1 by reacting with the compounds R²—X, wherein R² and X may have the abovementioned meanings. This synthesis step may also be carried out analogously to the examples of synthesis disclosed in WO 92/16528. In the case wherein R¹ and R² together form an alkylene bridge there is no need to add the reagent R²—X, as will be apparent to the skilled man. In this case the compounds of formula 4 contain a suitably substituted group R¹ (for example —C₃-C₅-alkylene-halogen) according to the above definitions and the compounds of formula 1 are prepared by intramolecular quaternisation of the amine.

Alternatively to the method of synthesising the compounds of formula 4 illustrated in Diagram 1 the derivatives 4 wherein the nitrogen bicyclic group denotes a scopine derivative may be obtained by oxidation (epoxidation) of compounds of formula 4 wherein the nitrogen bicyclic group is a tropenyl group. The following procedure may be used, according to the invention.

The compound 4 wherein A denotes —CH═CH— is suspended in a polar organic solvent, preferably in a solvent selected from among N-methyl-2-pyrrolidone (NMP), dimethylacetamide and dimethylformamide, preferably dimethylformamide, and then heated to a temperature of about 30-90° C., preferably 40-70° C. Then a suitable oxidising agent is added and the mixture is stirred at constant temperature for 2 to 8 hours, preferably 3 to 6 hours. The preferred oxidant is preferably vanadium pentoxide admixed with H₂O₂, most preferably H₂O₂-urea complex in combination with vanadium pentoxide. The product is worked up in the usual way. The products may be purified by crystallisation or chromatography, depending on their crystallisation tendencies.

Alternatively, the compounds of formula 4 wherein R denotes halogen may also be prepared by the method shown in Diagram 2.

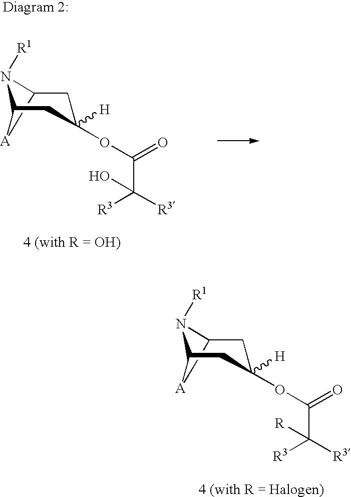

For this, the compounds of formula 4 wherein R denotes hydroxy are converted into the compounds 4 wherein R denotes halogen using suitable halogenation reagents. The method used for the halogenation reactions to be carried out according to Diagram 2 is sufficiently well known in the art.

As is apparent from Diagram 1, the intermediate products of general formula 4 have a central importance. Accordingly, in another aspect, the present invention relates to the intermediates of formula 4 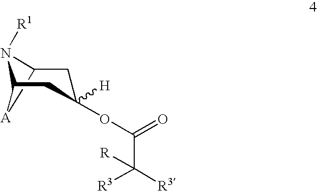
wherein the groups A, R, R¹, R³ and R^{3′} may be defined as above, optionally in the form of the acid addition salts thereof.

By acid addition salts are meant salts selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Preferably, according to the invention, the compounds of formula 4 are used as starting materials in the α-configured form. These α-configured compounds are therefore of particular importance according to the invention and correspond to general formula 4-α.

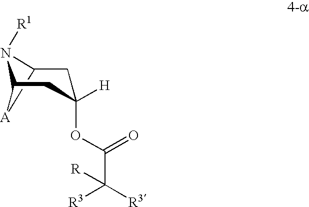

In another aspect the present invention relates to the use of compounds of general formula 2 for preparing the compounds of general formula 4. Moreover, the present invention relates to the use of the compounds of general formula 2 as starting materials for preparing the compounds of general formula 1. Furthermore, the present invention relates to the use of the compounds of general formula 4 as intermediate products for preparing the compounds of general formula 1.

The examples of synthesis described below serve to illustrate the present invention still further. However, they are to be regarded as only examples of the procedure, as further illustration of the invention, without restricting the invention to the object described below by way of example.

### EXAMPLE 1

### Tropenol di-(2-furyl)-glycolate methobromide;

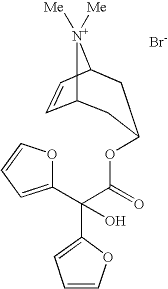 1.1.: Methyl di-(2-furyl)-glycolate (3a)

25.0 g (0.131 mol) alpha-furyl are added to a 15% KOH solution (100 g KOH flakes in 625 g water) at 80° C. with stirring. The mixture is stirred for a further 40 min. After cooling, 500 ml of diethyl ether are added and the mixture is acidified with 196 g (1.0 mol) 50% sulphuric acid added dropwise thereto, while being cooled with a cooling mixture of ice and methanol at a temperature of −5′ to +3° C. The organic phase is separated off and the mixture is again extracted with ice-cold diethyl ether. The combined diethyl ether phases are added at 0° C. to 800 ml (0.6 mol) of a freshly prepared solution of diazomethane in diethyl ether. The resulting mixture is stirred for another 90 min. and then slowly heated to ambient temperature. Inorganic matter is filtered off and the solution is evaporated to dryness.

To concentrate it further, it is combined with diethyl ether, the precipitate formed is filtered off and the solution is evaporated to dryness.

Yield: 18.0 g (=62% of theory).

1.2.: Tropenol di-(2-furyl)-glycolate (4a)

8.9 g (0.04 mol) of methyl di-(2-furyl)-glycolate 3a, 5.57 g (0.04 mol) of tropenol and 0.3 g of sodium are stirred for 3 h at an oil bath temperature of 80° C. under 80 mbar. After cooling to ambient temperature, acetonitrile is added until the sodium has finished reacting. The mixture is evaporated to dryness, the residue is extracted with dichloromethane and water, the organic phase is dried over magnesium sulphate and the solvent is distilled off. The residue is combined with diethyl ether, insoluble matter is filtered off and the solution is evaporated to dryness.

Yield: 3.25 g (=25% of theory)

1.3.: Tropenol di-(2-furyl)-glycolate methobromide

3.25 g (0.01 mol) of tropenol di-(2-furyl)-glycolate 4a are placed in 40 ml of acetonitrile and 30 ml of dichloromethane, combined with 4.0 g (0.017 mol) of 39.4% methyl bromide solution in acetonitrile, shaken and left to stand in the dark for 2 days. The solution is concentrated, the crystals formed are suction filtered, washed with ice-cold acetonitrile and dried. The crystals are heated to boiling in acetonitrile, treated with activated charcoal, filtered, concentrated and crystallised. Yield: 1.68 g (=40% of theory); melting point: 221°-222° C.

### EXAMPLE 2

### Scopine di-(2-furyl)-glycolate methobromide

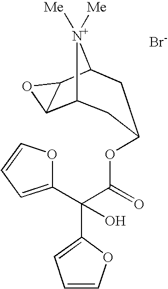 2.1.: Scopine di-(2-furyl)-glycolate (4b):

3.45 g (0.01 mol) of tropenol di-(2-furyl)-glycolate 4a and 0.2 g (0.001 mol) of vanadium-(V)-oxide are placed in 60 ml of dimethylformamide, heated to 55° C. and then combined with a solution of 1.98 g (0.021 mol) of H₂O₂-urea in 10 ml of water within 15 min. After 2 h the same amount of vanadium-(V)-oxide and H₂O₂-urea are added again and the mixture is then left to stand for 72 h at ambient temperature. It is diluted with water to a volume of 500 ml, extracted with dichloromethane, the organic phase is dried over magnesium sulphate and the solvent is distilled off. The residue is distributed between diethyl ether and water, the organic phase is dried over magnesium sulphate and evaporated to dryness. Yield: 0.93 g (=27% of theory).

2.2.: Scopine di-(2-furyl)-glycolate methobromide:

0.95 g (0.003 mol) of scopine di-(2-furyl)-glycolate are dissolved in 20 ml of acetonitrile and 10 ml of dichloromethane, combined with 3.0 g (0.013 mol) of 40% methyl bromide solution in acetonitrile and left to stand in the dark for 2 days. The mixture is filtered, the filtrate is concentrated until crystallisation sets in, then diluted with acetone, cooled, suction filtered, washed and dried.

Yield: 0.58 g (=44% of theory); melting point: 216° C.

It was found that the compounds of formula 1 according to the invention are antagonists of the M3 receptor (Muscarinic Receptor subtype 3). The compounds according to the invention have Ki values of less than 10 nM in terms of their affinity for the M3 receptor. These values were determined by the method described below.

Chemicals

3H-NMS was obtained from Messrs Amersham of Braunschweig, with a specific radioactivity of 3071 GBq/mmol (83 Ci/mmol). All the other reagents were obtained from Serva of Heidelberg and Merck of Darmstadt.

Cell Membranes:

We used cell membranes from CHO (Chinese hamster ovary) cells which were transfected with the corresponding genes of the human muscarinic receptor subtypes hml to hm5 (BONNER). The cell membranes of the desired subtype were thawed, resuspended by hand with a glass homogeniser and diluted with HEPES buffer to a final concentration of 20-30 mg of protein/ml.

Receptor Binding Studies:

The binding assay was carried out in a final volume of 1 ml and consisted of 100 μl of unlabelled substance in various concentrations, 100 μl of radioligand (3H—N-methylscopolamine 2 nmol/L (3H-NMS), 200 μl of membrane preparation and 600 μl of HEPES buffer (20 mmol/L HEPES, 10 mmol/L MgCl₂, 100 mmol/L NaCl, adjusted with 1 mol/L NaOH to pH 7.4).

The nonspecific binding was determined using 10 mmol/l of atropine.

The preparation was incubated for 45 min. at 37° C. in 96-well microtitre plates (Beckman, polystyrene, No. 267001) as a double measurement. The incubation was ended by filtering using an Inotech Cell Harvester (type IH 110) through Whatman G-7 filters. The filters were washed with 3 ml of ice-cooled HEPES buffer and dried before measuring.

Determining the Radioactivity:

The radioactivity of the filter mats was measured simultaneously using a two-dimensional digital autoradiograph (Berthold, Wildbad, type 3052).

Evaluation:

The Ki values were calculated using implicit equations which were derived directly from the mass-action law, with the model for the 1 receptor 2 ligand reaction (SysFit software, SCHITTKOWSKI).

Literature:

BONNER TI, New subtypes of muscarinic acetylcholine receptors Trends Pharmacol. Sci. 10, Suppl.: 11-15 (1989); SCHITTKOWSKI K Parameter estimation in systems of nonlinear equations Numer Math. 68: 129-142 (1994).

The compounds of formula 1 according to the invention are characterised by their range of uses in the therapeutic field. Particular mention should be made of those applications for which the compounds of formula 1 according to the invention may preferably be used on the basis of their pharmaceutical activity as anticholinergics.

These are for example the treatment of asthma or COPD (chronic obstructive pulmonary disease). The compounds of general formula 1 may also be used to treat vagally induced sinus bradycardia and to treat heart rhythm disorders. Generally, the compounds according to the invention may also be used therapeutically to treat spasms, for example, in the gastrointestinal tract. They may also be used to treat spasms in the urinary tract and also to treat menstrual pain, for example. Of the ranges of indications mentioned above, the treatment of asthma and COPD with the compounds of formula 1 according to the invention is of particular importance.

The compounds of general formula 1 may be used on their own or in conjunction with other active substances of formula 1. The compounds of general formula 1 may optionally also be used in combination with other pharmacologically active substances. These may be, in particular, betamimetics, antiallergics, PAF antagonists, PDE-IV inhibitors, leukotriene antagonists, p38 kinase inhibitors, EGFR kinase inhibitors and corticosteroids as well as combinations of active substances thereof.

Examples of betamimetics which may be used according to the invention in conjunction with the compounds of formula 1 include compounds selected from among bambuterol, bitolterol, carbuterol, clenbuterol, fenoterol, formoterol, hexoprenaline, ibuterol, pirbuterol, procaterol, reproterol, salmeterol, sulphonterol, terbutaline, tolubuterol, 4-hydroxy-7-[2-{[2-{[3-(2-phenylethoxy)propyl]sulphonyl}ethyl]-amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluormethylphenyl)-2-tert.-butylamino)ethanol and 1-(4-ethoxycarbonylamino-3-cyano-5-fluorophenyl)-2-(tert.-butylamino)ethanol, optionally in the form of the racemates, the enantiomers, the diastereomers and optionally the pharmacologically acceptable acid addition salts, the solvates and/or the hydrates thereof. Most preferably, the betamimetics used as active substances in conjunction with the compounds of formula 1 according to the invention are selected from among fenoterol, formoterol, salmeterol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, optionally in the form of the racemates, the enantiomers, the diastereomers and optionally the pharmacologically acceptable acid addition salts and the hydrates thereof. Of the betamimetics mentioned above the compounds formoterol and salmeterol are particularly preferred, optionally in the form of the racemates, the enantiomers, the diastereomers and optionally the pharmacologically acceptable acid addition salts thereof, and the hydrates thereof. According to the invention, the acid addition salts of the betamimetics selected, for example, from among the hydrochloride, hydrobromide, sulphate, phosphate, fumarate, methanesulphonate and xinafoate are preferred. Particularly preferred in the case of salmeterol are the salts selected from among the hydrochloride, sulphate and xinafoate, of which the xinafoate is particularly preferred. Particularly preferred in the case of formoterol are the salts selected from among the hydrochloride, sulphate and fumarate, of which the hydrochloride and fumarate are particularly preferred. According to the invention, formoterol fumarate is of exceptional importance.

Within the scope of the present invention, the corticosteroids which may optionally be used in conjunction with the compounds of formula 1 may be compounds selected from among flunisolide, beclomethasone, triamcinolone, budesonide, fluticasone, mometasone, ciclesonide, rofleponide, GW 215864, KSR 592, ST-126 and dexamethasone. Preferably, within the scope of the present invention, the corticosteroids are selected from among flunisolide, beclomethasone, triamcinolone, budesonide, fluticasone, mometasone, ciclesonide and dexamethasone, while budesonide, fluticasone, mometasone and ciclesonide are important and budesonide and fluticasone are particularly important. In some cases, within the scope of the present patent application, the term steroids is used on its own instead of the word corticosteroids. Any reference to steroids within the scope of the present invention includes a reference to salts or derivatives which may be formed from the steroids. Examples of possible salts or derivatives include: sodium salts, sulphobenzoates, phosphates, isonicotinates, acetates, propionates, dihydrogen phosphates, palmitates, pivalates or furoates. In some cases the corticosteroids may also occur in the form of their hydrates.

Examples of PDE-IV inhibitors which may be used according to the invention as a combination with the compound of formula 1 include compounds selected from among enprofylline, roflumilast, ariflo, Bay-198004, CP-325,366, BY343, D-4396 (Sch-351591), V-11294A and AWD-12-281. Preferred PDE-IV inhibitors are selected from among enprofylline, roflumilast, ariflo and AWD-12-281, while AWD-12-281 is particularly preferred as the combination partner with the compound of formula 1 according to the invention. Any reference to the abovementioned PDE-IV inhibitors also includes, within the scope of the present invention, a reference to any pharmacologically acceptable acid addition salts thereof which may exist. By the physiologically acceptable acid addition salts which may be formed by the abovementioned PDE-IV inhibitors are meant, according to the invention, pharmaceutically acceptable salts selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. According to the invention, the salts selected from among the acetate, hydrochloride, hydrobromide, sulphate, phosphate and methanesulphonate are preferred in this context.

Within the scope of the present invention, the term dopamine agonists, which may optionally be used in conjunction with the compounds of formula 1, denotes compounds selected from among bromocriptine, cabergolin, alpha-dihydroergocryptine, lisuride, pergolide, pramipexol, roxindol, ropinirol, talipexol, tergurid and viozan. It is preferable within the scope of the present invention to use, as combination partners with the compounds of formula 1, dopamine agonists selected from among pramipexol, talipexol and viozan, pramipexol being of particular importance. Any reference to the abovementioned dopamine agonists also includes, within the scope of the present invention, a reference to any pharmacologically acceptable acid addition salts and hydrates thereof which may exist. By the physiologically acceptable acid addition salts thereof which may be formed by the abovementioned dopamine agonists are meant, for example, pharmaceutically acceptable salts selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid.

Examples of antiallergic agents which may be used according to the invention as a combination with the compound of formula 1 include epinastin, cetirizin, azelastin, fexofenadin, levocabastin, loratadine, mizolastin, ketotifen, emedastin, dimetinden, clemastine, bamipin, cexchloropheniramine, pheniramine, doxylamine, chlorophenoxamine, dimenhydrinate, diphenhydramine, promethazine, ebastin, desloratidine and meclizine. Preferred antiallergic agents which may be used within the scope of the present invention in combination with the compounds of formula 1 according to the invention are selected from among epinastin, cetirizin, azelastin, fexofenadin, levocabastin, loratadine, ebastin, desloratidine and mizolastin, epinastin and desloratidine being particularly preferred. Any reference to the above-mentioned antiallergic agents also includes, within the scope of the present invention, a reference to any pharmacologically acceptable acid addition salts thereof which may exist.

Examples of PAF antagonists which may be used according to the invention as a combination with the compounds of formula 1 include 4-(2-chlorophenyl)-9-methyl-2-[3-(4-morpholinyl)-3-propanon-1-yl]-6H-thieno-[3,2-f][1,2,4]triazolo[4.3-a][1,4]diazepine, 6-(2-chlorophenyl)-8,9-dihydro-1-methyl-8-[(4-morpholinyl)carbonyl]-4H,7H-cyclo-penta-[4.5]thieno-[3,2-f] [1,2,4]triazolo[4,3-a][1,4]diazepine.

Examples of EGFR kinase inhibitors which may be used as a combination with the compounds of formula 1 according to the invention include, in particular, 4-[(3-chloro-4-fluoro-phenyl)amino]-7-[4-((R)-6-methyl-2-oxo-morpholin-4-yl)-butyloxy]-6-[(vinylcarbonyl)amino]-quinazoline, 4-[(3-chloro-4-fluoro-phenyl)amino]-7-[4-((S)-6-methyl-2-oxo-morpholin-4-yl)-butyloxy]-6-[(vinylcarbonyl)amino]-quinazoline, 4-[(3-chloro-4-fluoro-phenyl)amino]-7-(2-{4-[(S)-(2-oxo-tetrahydrofuran-5-yl)carbonyl]-piperazin-1-yl}-ethoxy)-6-[(vinylcarbonyl)amino]-quinazoline, 4-[(3-chloro-4-fluoro-phenyl)amino]-7-[2-((S)-6-methyl-2-oxo-morpholin-4-yl)-ethoxy]-6-[(vinylcarbonyl)amino]-quinazoline, 4-[(3-chloro-4-fluorophenyl)amino]-6-[(4-{N-[2-(ethoxycarbonyl)-ethyl]-N-[(ethoxycarbonyl)methyl]amino}-1-oxo-2-buten-1-yl)amino]-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopropylmethoxy-quinazoline and 4-[(3-chloro-4-fluorophenyl)amino]-6-[3-(morpholin-4-yl)-propyloxy]-7-methoxy-quinazoline. Any reference to the abovementioned EGFR kinase inhibitors also includes, within the scope of the present invention, a reference to any pharmacologically acceptable acid addition salts thereof which may exist. By the physiologically or pharmacologically acceptable acid addition salts thereof which may be formed by the EGFR kinase inhibitors are meant, according to the invention, pharmaceutically acceptable salts selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid. The salts of the EGFR kinase inhibitors selected from among the salts of acetic acid, hydrochloric acid, hydrobromic acid, sulphuric acid, phosphoric acid and methanesulphonic acid are preferred according to the invention.

Particularly preferred examples of p38 kinase inhibitors which may be used as a combination with the compounds of formula 1 according to the invention include 1-[5-tert-butyl-2-p-tolyl-2H-pyrazol-3-yl]-3-[4-(2-morpholin-4-yl-ethoxy)naphthalin-1-yl]-urea; 1-[5-tert-butyl-2-p-tolyl-2H-pyrazol-3-yl]-3-[4-(2-(1-oxothiomorpholin-4-yl)ethoxy)naphthalin-1-yl]-urea; 1-[5-tert-butyl-2-(2-methylpyridin-5-yl)-2H-pyrazol-3-yl]-3-[4-(2-pyridine-4-yl-ethoxy)naphthalin-1-yl]-urea; 1-[5-tert-butyl-2-(2-methoxypyridin-5-yl)-2H-pyrazol-3-yl]-3-[4-(2-morpholin-4-yl-ethoxy)naphthalin-1-yl]-urea or 1-[5-tert-butyl-2-methyl-2H-pyrazol-3-yl]-3-[4-(2-morpholin-4-yl-ethoxy)naphthalen-1-yl]-urea. Any reference to the abovementioned p38 kinase inhibitors also includes, within the scope of the present invention, a reference to any pharmacologically acceptable acid addition salts thereof which may exist. By the physiologically or pharmacologically acceptable acid addition salts thereof which may be formed by the p38 kinase inhibitors are meant, according to the invention, pharmaceutically acceptable salts selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid.

If the compounds of formula 1 are used in conjunction with other active substances, the combination with steroids, PDE IV inhibitors or betamimetics is particularly preferred, of the categories of compounds mentioned above. The combination with betamimetics, particularly with long-acting betamimetics, is of particular importance. The combination of the compounds of formula 1 according to the invention with salmeterol or formoterol is particularly preferred.

Suitable preparations for administering the salts of formula 1 include for example tablets, capsules, suppositories and solutions, etc. Administration of the compounds according to the invention by inhalation is of particular importance according to the invention (particularly for treating asthma or COPD). The content of the pharmaceutically active compound(s) should be in the range from 0.05 to 90 wt.-%, preferably 0.1 to 50 wt.-% of the composition as a whole. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates or stabilisers such as alkali metal salts of ethylenediaminetetraacetic acid, optionally using emulsifiers and/or dispersants, while if water is used as diluent, for example, organic solvents may optionally be used as solubilisers or dissolving aids, and the solutions may be transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, preferably potato starch, gelatin and the like. Lubricants such as magnesium stearate, sodium laurylsulphate and talc may also be used to produce the tablets. In the case of aqueous suspensions the active substances may be combined with various flavour enhancers or colourings in addition to the abovementioned excipients.

For administering the compounds of formula 1 for the treatment of asthma or COPD it is particularly preferred according to the invention to use preparations or pharmaceutical formulations which are suitable for inhalation. Inhalable preparations include inhalable powders, propellant-containing metering aerosols or propellant-free inhalable solutions Within the scope of the present invention, the term propellant-free inhalable solutions also includes concentrates or sterile inhalable solutions ready for use. The formulations which may be used within the scope of the present invention are described in more detail in the next part of the specification

The inhalable powders which may be used according to the invention may contain 1 either on their own or in admixture with suitable physiologically acceptable excipients.

If the active substances 1 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipient mentioned above. These finer excipients are also selected from the group of possible excipients listed hereinbefore. Finally, in order to prepare the inhalable powders according to the invention, micronised active substance 1, preferably with an average particle size of 0.5 to 10 μm, more preferably from 1 to 5 μm, is added to the excipient mixture. Processes for producing the inhalable powders according to the invention by grinding and micronising and finally mixing the ingredients together are known from the prior art. The inhalable powders according to the invention may be administered using inhalers known from the prior art.

The inhalation aerosols containing propellant gas according to the invention may contain the compounds 1 dissolved in the propellant gas or in dispersed form. The compounds 1 may be contained in separate formulations or in a common formulation, in which the compounds 1 are either both dissolved, both dispersed or in each case only one component is dissolved and the other is dispersed. The propellant gases which may be used to prepare the inhalation aerosols are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The abovementioned propellant gases may be used either on their own or in mixtures thereof. Particularly preferred propellant gases are halogenated alkane derivatives selected from TG134a and TG227 and mixtures thereof.

The propellant-driven inhalation aerosols may also contain other ingredients such as co-solvents, stabilisers, surfactants, antioxidants, lubricants and pH adjusters. All these ingredients are known in the art.

The propellant-driven inhalation aerosols according to the invention mentioned above may be administered using inhalers known in the art (MDIs=metered dose inhalers).

Moreover, the active substances 1 according to the invention may be administered in the form of propellant-free inhalable solutions and suspensions. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

The addition of editic acid (EDTA) or one of the known salts thereof, sodium edetate, as stabiliser or complexing agent may optionally be omitted in these formulations. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the physiologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the active substance 1, only benzalkonium chloride and sodium edetate. In another preferred embodiment, no sodium edetate is present.

The dosage of the compounds according to the invention is naturally highly dependent on the method of administration and the complaint which is being treated. When administered by inhalation the compounds of formula 1 are characterised by a high potency even at doses in the μg range. The compounds of formula 1 may also be used effectively above the μg range. The dosage may then be in the gram range, for example. Particularly when administered by routes other than by inhalation the compounds according to the invention may be administered in higher doses (for example, but not restrictively, in the range from 1 to 1000 mg).

The following examples of formulations illustrate the present invention without restricting its scope:

Examples of Pharmaceutical Formulations

| A) Tablets | per tablet |
|---|---|
| active substance 1 | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) Tablets | per tablet |
|---|---|
| active substance 1 | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium carboxymethyl starch and the magnesium stearate are added and mixed in and the mixture is compressed to form tablets of a suitable size.

| C) Ampoule solution | |
|---|---|
| active substance 1 | 50 mg |
| sodium chloride | 50 mg |
| water for inj. | 5 ml |

The active substance is dissolved in water at its own pH or optionally at pH 5.5 to 6.5 and sodium chloride is added to make the solution isotonic. The resulting solution is filtered to remove pyrogens and the filtrate is transferred under aseptic conditions into ampoules which are then sterilised and heat-sealed. The ampoules contain 5 mg, 25 mg and 50 mg of active substance.

| D) Metering aerosol | |
|---|---|
| active substance 1 | 0.005 |
| Sorbitan trioleate | 0.1 |
| Monofluorotrichloromethane and | ad 100 |
| Difluorodichloromethane 2:3 | |

The suspension is transferred into a conventional aerosol container with metering valve. Preferably 50 μl suspension are released on each actuation. The active substance may also be released in higher doses if desired (e.g. 0.02 wt.-%).

| E) Solutions (in mg/100 ml) | |
|---|---|
| active substance 1 | 333.3 mg |
| formoterol fumarate | 333.3 mg |
| benzalkonium chloride | 10.0 mg |
| EDTA | 50.0 mg |
| HCl(1n) ad pH | 3.4 |

This solution may be prepared in the usual way.

| F) Inhalable powder | |
|---|---|
| active substance 1 | 6 μg |
| formoterol fumarate | 6 μg |
| lactose monohydrate ad | 25 mg |

The inhalable powder is prepared in the usual way by mixing the individual ingredients.

| G) Inhalable powder | |
|---|---|
| active substance 1 | 10 μg |
| lactose monohydrate ad | 5 mg |

The inhalable powder is prepared in the usual way by mixing the individual ingredients.

1. A compound of the formula 1 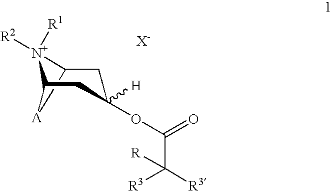
wherein
A denotes a double-bonded group selected from the group consisting of: 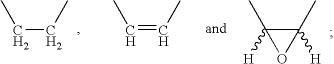
X⁻ denotes an anion with a single negative charge;
R denotes hydrogen, hydroxy, methyl, ethyl, —CF3, CHF2 or fluorine;
R¹ and R², which may be identical or different, denote —C1-C5-alkyl, which may optionally be substituted by —C3-C6-cycloalkyl, hydroxy, or halogen, or
R¹ and R² together denote a —C3-C5-alkylene bridge; and,
R³ and R^{3′} which may be identical or different, denote furyl, which may optionally be mono-, di- or trisubstituted by a group selected from the group consisting of: —C1-C4-alkyl, —C1-C4-alkyloxy, hydroxy, —CF3, —CHF2, CN, NO2, and halogen.

2. A compound of the formula 1 according to claim 1, wherein
A denotes a double-bonded group selected from the group consisting of: 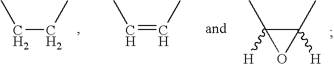
X⁻ denotes an anion with a single negative charge selected from the group consisting of: chloride, bromide, 4-toluenesulphonate, and methanesulphonate;
R denotes hydroxy, methyl, or fluorine;
R¹ and R², which may be identical or different, denote methyl, ethyl, or fluoroethyl; and,
R³ and R^{3′}, which may be identical or different, denote furyl, which may optionally be mono- or disubstituted by a group selected from the group consisting of: methyl, methyloxy, hydroxy, —CF3, —CHF2, fluorine, and chlorine.

3. A compound of the formula 1 according to claim 1, wherein
A denotes a double-bonded group selected from the group consisting of: 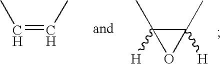
X⁻ denotes an anion with a single negative charge selected from the group consisting of: chloride, bromide, and methanesulphonate;
R denotes hydroxy, methyl, or fluorine;
R¹ and R², which may be identical or different, denote methyl or ethyl; and,
R³ and R^{3′} which may be identical or different, denote furyl, which may optionally be mono- or disubstituted by a group selected from the group consisting of: —CF3, —CHF2 and fluorine.

4. A compound of the formula 1 according to claim 1, wherein
A denotes a double-bonded group selected from the group consisting of: 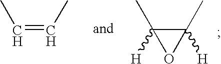
X⁻ denotes bromide;
R denotes hydroxy or methyl;
R¹ and R², which may be identical or different, denote methyl or ethyl; and,
R³ and R^{3′} which may be identical or different, denote furyl, which may optionally be mono- or disubstituted by fluorine.

5. A pharmaceutical composition comprising a compound according to claim 1, and a pharmaceutically acceptable carrier or excipient.

6. A pharmaceutical composition in accordance with claim 5, further comprising at least one other active substance selected from the group consisting of: the betamimetics, antiallergics, PAF antagonists, leukotriene antagonists, and steroids.

7. A method for treating asthma, COPD, vagally induced sinus bradycardia, heart rhythm disorders, spasms in the gastrointestinal tract, spasms in the urinary tract, or menstrual pain, the method comprising the step of administering to a host in need of such treatment a therapeutically effective amount of a compound of the formula 1 according to claim 1.

8. The method of claim 7, wherein there is further administered at least one other active substance selected from the group consisting of: the betamimetics, antiallergics, PAF antagonists, leukotriene antagonists, and steroids.

9. A compound of the formula 4 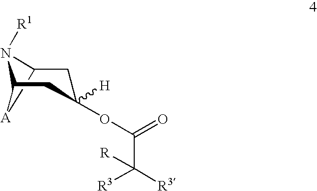
wherein:
A denotes a double-bonded group selected from the group consisting of: 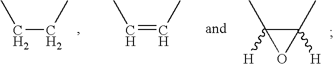
R denotes hydrogen, hydroxy, methyl, ethyl, —CF3, CHF2, or fluorine;
R¹ denotes —C1-C5-alkyl, which may optionally be substituted by —C3-C6-cycloalkyl, hydroxy, or halogen; and,
R³ and R^{3′} which may be identical or different, denote furyl, which may optionally be mono-, di- or trisubstituted by a group selected from the group consisting of: —C1-C4-alkyl, —C1-C4-alkyloxy, hydroxy, —CF3, —CHF2, CN, NO2, and halogen; or an acid addition salt thereof.

10. A compound of the formula 4 according to claim 9, wherein
A denotes a double-bonded group selected from the group consisting of: 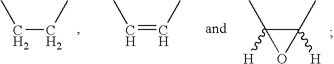
R denotes hydroxy, methyl, or fluorine;
R¹ denotes methyl, ethyl, or fluoroethyl; and,
R³ and R^{3′} which may be identical or different, denote furyl, which may optionally be mono- or disubstituted by a group selected from the group consisting of methyl, methyloxy, hydroxy, —CF3, —CHF2, fluorine, and chlorine;
or an acid addition salt thereof.

11. A compound of the formula 4 according to claim 9, wherein
A denotes a double-bonded group selected from the group consisting of: 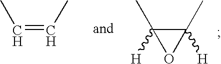
R denotes hydroxy, methyl, or fluorine;
R1 denotes methyl or ethyl; and,
R³ and R^{3′}, which may be identical or different, denote furyl, which may optionally be mono- or disubstituted by a group selected from the group consisting of: —CF3, —CHF2, and fluorine;
or an acid addition salt thereof.

12. A compound of the formula 4 according to claim 9, wherein
A denotes a double-bonded group selected from the group consisting of 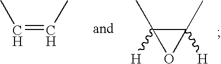
R denotes hydroxy or methyl;
R¹ denotes methyl or ethyl; and,
R³ and R^{3′} which may be identical or different, denote furyl, which may optionally be mono- or disubstituted by fluorine;
or an acid addition salt thereof.

13. A pharmaceutical composition comprising a compound according to claim 2 and a pharmaceutically acceptable carrier or excipient.

14. A pharmaceutical composition comprising a compound according to claim 3 and a pharmaceutically acceptable carrier or excipient.

15. A pharmaceutical composition comprising a compound according to claim 4 and a pharmaceutically acceptable carrier or excipient.

16. A method for treating asthma, COPD, vagally induced sinus bradycardia, heart rhythm disorders, spasms in the gastrointestinal tract, spasms in the urinary tract, or menstrual pain, the method comprising the step of administering to a host in need of such treatment a therapeutically effective amount of a compound of the formula 1 according to claim 2.

17. A method for treating asthma, COPD, vagally induced sinus bradycardia, heart rhythm disorders, spasms in the gastrointestinal tract, spasms in the urinary tract, or menstrual pain, the method comprising the step of administering to a host in need of such treatment a therapeutically effective amount of a compound of the formula 1 according to claim 3.

18. A method for treating asthma, COPD, vagally induced sinus bradycardia, heart rhythm disorders, spasms in the gastrointestinal tract, spasms in the urinary tract, or menstrual pain, the method comprising the step of administering to a host in need of such treatment a therapeutically effective amount of a compound of the formula 1 according to claim 4.
